(19) United States
(12) Patent Application Publication
(10) Pub. No.: US 2024/0301488 A1
(43) Pub. Date: Sep. 12, 2024

(54) METHODS FOR IDENTIFYING A LOCATION OF AN RNA IN A BIOLOGICAL SAMPLE

(71) Applicant: 10x Genomics, Inc., Pleasanton, CA (US)

(72) Inventor: Marlon Stoeckius, Stockholm (SE)

(73) Assignee: 10x Genomics, Inc., Pleasanton, CA (US)

(21) Appl. No.: 18/605,197

(22) Filed: Mar. 14, 2024

Related U.S. Application Data
(63) Continuation of application No. 18/448,618, filed on Aug. 11, 2023, now Pat. No. 11,952,627.
Application No. 18/448,618 is a continuation of application No. 17/368,276, filed on Jul. 6, 2021, now Pat. No. 11,761,038.
(60) Provisional application No. 63/048,627, filed on Jul. 6, 2020.

(51) Int. Cl. C12Q 1/6874; C12Q 1/48; C12Q 1/6806; C12Q 1/686; C12Q 1/6876

(52) U.S. Cl. CPC C12Q 1/6874; C12Q 1/485; C12Q 1/6806; C12Q 1/686; C12Q 1/6876; C12Y 207/07007; C12Y 301/26004; C12Y 605/01001; G01N 2333/9015; G01N 2333/9126; G01N 2333/922

(57) ABSTRACT

Provided herein are methods of identifying a location of an RNA in a sample that include: (a) contacting the sample with an array comprising capture probes, where a capture probe comprises a capture domain and a spatial barcode; (b) releasing the RNA from the sample; (c) extending a 3′ end of the capture probe using the capture domain-bound RNA as a template; (d) generating nick(s) in the extended capture probe-hybridized RNA and performing random-primed DNA synthesis; (e) performing end repair on the second strand DNA molecule; (f) adding a single adenosine nucleotide to the 3′ end of the extended capture probe; (g) ligating a double-stranded sequencing adaptor to the double-stranded DNA product; and (h) determining all or a part of the sequence of the RNA, and the sequence of the spatial barcode, or complements thereof, and using the determined sequences to identify the location of the RNA in the sample.

### CROSS-REFERENCE TO RELATED APPLICATIONS

This application is a continuation of U.S. patent application Ser. No. 18/448,618, filed on Aug. 11, 2023, which is a continuation of U.S. patent application Ser. No. 17/368,276, now U.S. Pat. No. 11,761,038, filed Jul. 6, 2021, which claims priority to U.S. Provisional Patent Application No. 63/048,627, filed on Jul. 6, 2020, the contents of each of which are incorporated herein by reference in its entirety.

### BACKGROUND

Cells within a tissue have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, signaling, and cross-talk with other cells in the tissue.

Spatial heterogeneity has been previously studied using techniques that typically provide data for a handful of analytes in the context of intact tissue or a portion of a tissue (e.g., tissue section), or provide significant analyte data from individual, single cells, but fails to provide information regarding the position of the single cells from the originating biological sample (e.g., tissue).

RNA sequencing libraries generated from tissue samples can pose some challenges. Methods, reaction mixtures, and kits to insert a sequencing adapter directly to the second-strand DNA, thereby adding a second sequencing adapter to a synthesized cDNA previously generated directly on the spatial array would increase efficiency.

### SUMMARY

Methods for improving the efficiency of adding a sequencing oligonucleotide to a 3′ end of a cDNA generated using a spatial array are desired. The present invention is based on the discovery of an improved method for adding a sequencing oligonucleotide to a 3′ end of a cDNA generated on a spatial array that includes the use of RNAse H or a functional equivalent to produce nicks in the RNA of a RNA:DNA hybrid preceding second strand synthesis followed by end repair, A-tailing, and ligation of a double-stranded adaptor to a blunt-ended double-stranded DNA product. The ligation of a double-stranded adaptor to the blunt-ended double-stranded DNA product is highly efficient, comparative to alternative methods for adaptor incorporation, and contributes to the higher sensitivity of the methods provided herein. The disclosed methods can decrease the hands on time of a researcher in adding a sequencing oligonucleotide to a 3′ end of a cDNA generated on a spatial array (e.g., the methods described herein can be performed within about 12 hours as compared to alternative methods of adaptor incorporation which can take up to one day).

Thus, provided herein are methods of identifying a location of an RNA in a biological sample, the method including: (a) contacting the biological sample with an array including a plurality of capture probes, where a capture probe of the plurality of capture probes includes a capture domain and a spatial barcode; (b) releasing the RNA from the biological sample, where the RNA released from the biological sample is specifically bound by the capture domain of the capture probe; (c) extending a 3′ end of the capture probe using the RNA bound by the capture domain as a template, thereby generating an extended capture probe hybridized to the RNA; (d) generating one or more nick(s) in the RNA hybridized to the extended capture probe and performing random-primed DNA synthesis, thereby replacing the nicked RNA with a second strand DNA molecule hybridized to the extended capture probe; (e) performing end repair on the second strand DNA molecule hybridized to the extended capture probe to generate a blunt-ended double-stranded DNA product; (f) adding a single adenosine (A) nucleotide to the 3′ end of the extended capture probe in the blunt-ended double-stranded DNA product; (g) ligating a double-stranded sequencing adaptor to the double-stranded DNA product; and (h) determining (i) all or a part of the sequence of the RNA or a complement thereof, or (ii) all or a part of the sequence of the spatial barcode or a complement thereof, and using the determined sequences of (i) and (ii) to identify the location of the RNA in the biological sample.

In some embodiments, generating of the one or more nick(s) in step (d) is performed using RNAse H. In some embodiments, the random-primed DNA synthesis is performed using DNA polymerase I.

In some embodiments, the method includes: (i) the end repair in step (e) is performed using a Klenow fragment and/or T4 DNA polymerase; (ii) step (f) is performed using Taq polymerase; and (iii) step (g) is performed using a kinase, where the kinas is a polynucleotide kinase and a DNA ligase.

In some embodiments, the RNA is a mRNA.

In some embodiments, the method includes: (i) the capture domain is positioned 3′ relative to the spatial barcode in the capture probe and where the capture domain includes a poly(T) sequence; and (ii) the capture probe includes a unique molecular identifier and/or a cleavage domain.

In some embodiments, the method includes between steps (g) and (h), a step of releasing the blunt-ended double-stranded DNA product from the array via cleavage of the cleavage domain.

In some embodiments, the determining in step (h) includes sequencing (i) all or a part of the sequence of the RNA or a complement thereof, or (ii) all or a part of the sequence of the spatial barcode or a complement thereof, where the sequencing is high throughput sequencing, where the high throughput sequencing includes sequencing by ligation or hybridization of labelled probes.

In some embodiments, the method includes creating a library for sequencing from the adaptor-ligated double-stranded DNA product, where the step of creating the library includes amplification, where the amplification includes polymerase chain reaction and the method includes sequencing the library.

In some embodiments, the releasing in step (b), includes permeabilizing the biological sample.

In some embodiments, the array is a slide, where the slide includes beads or wells.

In some embodiments, the biological sample is a tissue sample, a tissue section or a fixed tissue section, and optionally where the fixed tissue section is a formalin-fixed paraffin-embedded tissue section or the tissue section is a fresh, frozen tissue section.

In some embodiments, the method includes one or both of staining and imaging the biological sample.

Also provided herein are reaction mixtures including: an array including a plurality of capture probes, where a capture probe of the plurality includes (i) a spatial barcode and (ii) a capture domain that binds to an RNA; RNAse H; DNA polymerase I; DNA ligase; and a double-stranded sequencing adaptor.

In some embodiments, the reaction mixture includes: (i) the capture domain is positioned 3′ of the spatial barcode in the capture domain; and (ii) the capture probe includes a unique molecular identifier and/or a cleavage domain.

In some embodiments, the reaction mixture includes an RNA from a biological sample and includes one or more of Taq polymerase, a polynucleotide kinase (PNK), a reverse transcriptase, a Klenow fragment, and T4 DNA polymerase.

Also provided herein are kits including: an array including a plurality of capture probes, where a capture probe of the plurality includes (i) a spatial barcode and (ii) a capture domain that binds to an RNA; RNAse H; DNA polymerase I; DNA ligase; and a double-stranded sequencing adaptor.

In some embodiments, the kit includes: (i) the capture domain is positioned 3′ of the spatial barcode in the capture domain; and (ii) the capture probe includes a unique molecular identifier and/or a cleavage domain.

In some embodiments, the array is a slide, where the slide includes beads or wells.

In some embodiments, the kit includes one or more of Taq polymerase, a polynucleotide kinase (PNK), a reverse transcriptase, a Klenow fragment, and T4 DNA polymerase.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise. Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2A shows mRNA molecules captured by capture probes affixed to the surface of a spatial array.

FIG. 2B shows target analyte capture by a capture domain.

FIG. 2C shows mRNA captured by a capture domain, where the capture domain is used as a primer for extension to generate an extended capture probe using RNA specifically-bound to the capture domain as a template.

FIG. 2D shows an RNase utilized to create nicks in an RNA hybridized to the extended capture probe, thereby furnishing RNA primers access for use in random-primed DNA synthesis using, for example, DNA polymerase I, thereby replacing the nicked RNA with a second strand DNA molecule hybridized to the extended capture probe.

FIG. 2E shows end repair performed on the second strand DNA molecule hybridized to the extended capture probe to generate a blunt-ended double-stranded DNA product, and a single adenosine (A) nucleotide added to the 3′ end of the extended capture probe (e.g., A-tailing) of the blunt-ended double-stranded DNA product creating a one-nucleotide overhang.

FIG. 2F shows a double-stranded sequencing adaptor that is ligated to the blunt-ended double-stranded DNA product.

FIG. 2G shows a double-stranded DNA molecule that includes sequencing adaptors on opposite ends of the captured and double stranded DNA affixed to the spatial array.

### DETAILED DESCRIPTION

Capturing and sequencing as many target analytes from a spatial array is key in determining spatial gene expression in a biological sample. While current methods provide spatial gene expression data certain aspects of the workflow, for example, generating sequence ready libraries from a captured analyte, these methods can be inefficient resulting in a loss of sensitivity and specificity. For example, a certain percentage of the captured analytes can remain unamplified and therefore not sequenced due to workflow inefficiencies resulting in loss of gene expression data. Increasing the efficiencies of such workflows could result in a greater number of captured analytes being amplified and subsequently sequenced, thereby creating a more accurate spatial gene expression picture of a biological sample. The methods disclosed herein provide alternatives to creating sequencing ready libraries from captured analytes on a spatial array by increasing the number of analytes that are processed from library preparation through sequencing from a biological sample, thereby increasing the sensitivity and specificity of a spatial transcriptome array.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363(6434): 1463-1467, 2019; Lee et al., Nat. Protoc. 10(3):442-458, 2015; Trejo et al., PLOS ONE 14(2):e0212031, 2019; Chen et al., Science 348(6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

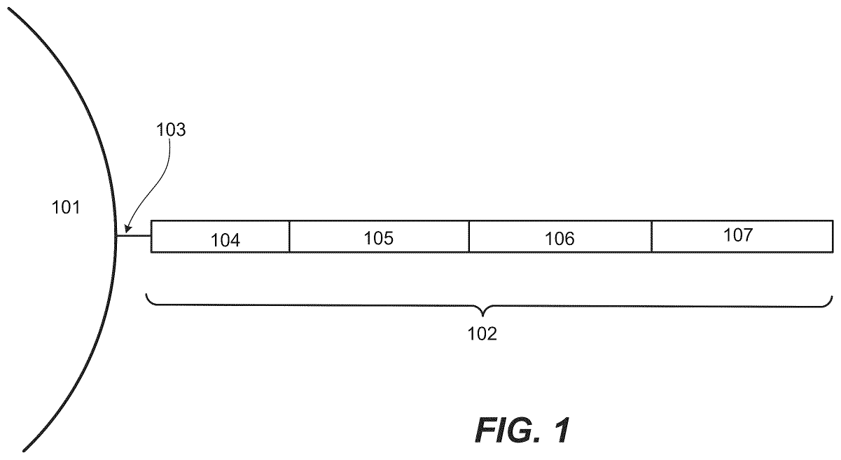

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that are useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. In some embodiments, the capture probe comprises one or more additional functional sequences that can be located, for example between the spatial barcode 105 and the UMI sequence 106, between the UMI sequence 106 and the capture domain 107, or following the capture domain 107. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 is common to all of the probes attached to a given feature. In some embodiments, the UMI sequence 106 of a capture probe attached to a given feature is different from the UMI sequence of a different capture probe attached to the given feature.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Methods for Increasing the Efficiency of Library Preparation of Nucleic Acids from a Spatial Array

Capturing and sequencing as many target analytes from a spatial array is key in determining spatial gene expression in a biological sample. While current methods provide spatial gene expression data certain aspects of the workflow, for example, generating sequence ready libraries from a captured analyte, can be inefficient resulting in a loss of sensitivity and specificity. For example, a certain percentage of the captured analytes can remain unamplified and therefore not sequenced due to workflow inefficiencies resulting in loss of gene expression data. Increasing the efficiencies of such workflows could result in a greater number of captured analytes being amplified and subsequently sequenced thereby creating a more accurate spatial gene expression picture of a biological sample. The methods disclosed herein provide alternatives to creating sequencing ready libraries from captured analytes on a spatial array by increasing the number of analytes that are processed from library preparation through sequencing from a biological sample, thereby increasing the sensitivity and specificity of a spatial transcriptome array.

The present invention is based on the discovery of an improved method for adding a sequencing oligonucleotide to a 3′ end of a cDNA generated on a spatial array that includes the use of RNAse H or a functional equivalent to produce nicks in the RNA of a RNA:DNA hybrid preceding second strand synthesis followed by end repair, A-tailing, and ligation of a double-stranded adaptor to a blunt-ended double-stranded DNA product. The ligation of a double-stranded adaptor to the blunt-ended double-stranded DNA product is highly efficient, relative to alternative methods for adaptor incorporation, and contributes to the higher sensitivity of the methods provided herein. The disclosed methods can decrease the hands-on time of a researcher in adding a sequencing oligonucleotide to a 3′ end of a cDNA generated on a spatial array (e.g., the methods described herein can be performed within about 12 hours as compared to alternative methods of adaptor incorporation which can take up to one day).

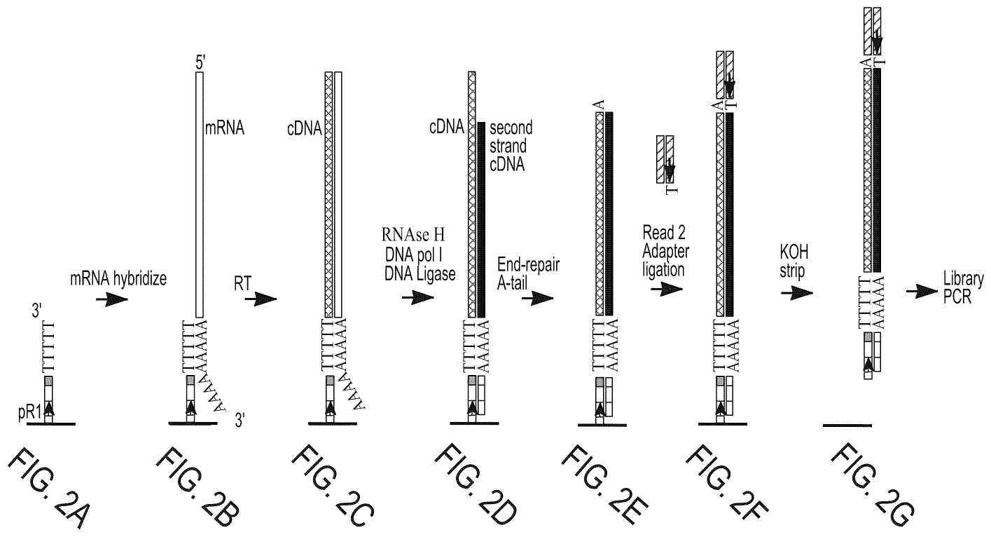

A schematic showing an exemplary embodiment of the methods provided herein is shown in FIGS. 2A-2G. The method includes disposing a biological sample, such as a tissue sample or section, on a spatial array and permeabilizing the biological sample to release mRNA molecules. After permeabilization, mRNA molecules are captured by capture probes affixed to the surface of the spatial array (FIG. 2A). The capture probes can include a capture domain (e.g., a polyT sequence), a spatial barcode, a unique molecular identifier (UMI), and a sequence for downstream sequencing (e.g., pR1). Upon target analyte capture by the capture domain (FIG. 2B), in this case mRNA, the capture domain(s) can be used as a primer for extension to generate an extended capture probe using RNA specifically-bound to the capture domain as a template (FIG. 2C). An RNase (e.g., RNase H) is utilized to create nicks in the RNA hybridized to the extended capture probe, thereby furnishing RNA primers access for use in random-primed DNA synthesis using, for example, DNA polymerase I, thereby replacing the nicked RNA with a second strand DNA molecule hybridized to the extended capture probe (FIG. 2D). End repair is performed on the second strand DNA molecule hybridized to the extended capture probe to generate a blunt-ended double-stranded DNA product (FIG. 2E). A single adenosine (A) nucleotide is added to the 3′ end of the extended capture probe (e.g., A-tailing) of the blunt-ended double-stranded DNA product creating a one-nucleotide overhang (FIG. 2E), wherein a double-stranded sequencing adaptor (Read 2 Adaptor) can be ligated (FIG. 2F), thereby creating a double-stranded DNA molecule that includes sequencing adaptors (e.g., pR1 and Read 2), on opposite ends of the captured and double stranded DNA affixed to the spatial array (FIG. 2G). The double-stranded DNA with the flanking sequencing adaptors can be released from the surface of the spatial array, by mechanical, chemical, or enzymatic means, and optionally, amplified to create a library (e.g., a spatial library) for sequencing. For example, a method for releasing the double stranded DNA with the flanking sequencing adaptors from the substrate, a combination of a salt, detergent (e.g., KOH, Tween, etc.) and heat can effect that release from the substrate. In some embodiments, heating includes heating from about 60° C. to about 90° C., from about 65° C. to about 85° C., or from about 70° C. to about 80° C. Alternative to double-stranded DNA release as shown in FIG. 2G, the extended capture probe remains affixed to the substrate while the second strand DNA is denatured from the extended capture probe, for example by a salt (e.g., KOH) alone as found in the Visium™ Spatial Gene Expression User Guide (CG000239). While the released DNA products from the array (e.g., spatial array) could be potentially sequenced without amplification, oftentimes additional amplification of library fragments is undertaken to increase the amount of sequenceable product from the array (e.g., spatial array). Following these steps, the location of the RNA in the biological sample can be identified using (i) all or a part of the sequence of the RNA or a complement thereof, or (ii) all or a part of the sequence of the spatial barcode or a complement thereof.

Thus, provided herein are methods of identifying a location of an RNA (e.g., mRNA or any of the other exemplary types of RNAs described herein or known in the art) in a biological sample (e.g., any of the exemplary biological samples described herein) that include: (a) contacting the biological sample with an array (e.g., any of the exemplary arrays described herein) comprising a plurality of capture probes, wherein a capture probe of the plurality of capture probes comprises a capture domain (e.g., any of the exemplary capture domains described herein) and a spatial barcode; (b) releasing the RNA from the biological sample, where the RNA released from the biological sample is bound by the capture domain of the capture probe; (c) extending a 3′ end of the capture probe using the RNA bound by the capture domain as a template, thereby generating an extended capture probe hybridized to the RNA; (d) generating one or more nick(s) in the RNA hybridized to the extended capture probe and performing random-primed DNA synthesis (e.g., using DNA polymerase I, and optionally, a DNA ligase), thereby replacing the nicked RNA with a second strand DNA molecule hybridized to the extended capture probe; (e) performing end repair on the second strand DNA molecule hybridized to the extended capture probe to generate a blunt-ended double-stranded DNA product (e.g., using a Klenow fragment and/or a T4 DNA polymerase); (f) adding a single adenosine (A) nucleotide to the 3′ end of the extended capture probe in the blunt-ended double-stranded DNA product (e.g., using Taq polymerase); (g) ligating a double-stranded sequencing adaptor to the double-stranded DNA product (e.g., using a kinase, e.g., a polynucleotide kinase, and a DNA ligase); and (h) determining (i) all or a part of the sequence of the RNA or a complement thereof, or (ii) all or a part of the sequence of the spatial barcode or a complement thereof, and using the determined sequences of (i) and (ii) to identify the location of the RNA in the biological sample.

A double-stranded adaptor sequencing adaptor can be any suitable double-stranded sequencing adaptor that can be used to perform any of the exemplary sequencing methods described herein. In some embodiments, the double-stranded sequencing adaptor can include an Illumina sequencing adaptor sequence. In some embodiments, the double-stranded sequencing adaptor can include a first and a second strand that each can be about 15 to 45 nucleotides (e.g., about 15 to about 45 nucleotides, about 15 to about 40 nucleotides, about 15 to about 35 nucleotides, about 15 to about 30 nucleotides, about 15 to about 25 nucleotides, or about 15 to about 20 nucleotides, about 20 to about 45 nucleotides, about 20 to about 40 nucleotides, about 20 to about 35 nucleotides, about 20 to about 30 nucleotides, about 20 to about 25 nucleotides, about 25 to about 45 nucleotides, about 25 to about 40 nucleotides, about 25 to about 35 nucleotides, about 25 to about 30 nucleotides, about 30 to about 45 nucleotides, about 30 to about 40 nucleotides, about 30 to about 35 nucleotides, about 35 to about 45 nucleotides, about 35 to about 40 nucleotides, or about 40 to about 45 nucleotides) long. In some embodiments, the double-stranded sequencing adaptor can include a universal sequence.

In some embodiments of any of the methods described herein, the capture domain comprises a poly(T) sequence. In some embodiments, the capture domain does not comprise a poly(T) sequence. In some embodiments, the capture domain comprises a sequence that is substantially complementary to a contiguous sequence present in the RNA. The capture domain can be about 5 to about 40 nucleotides (e.g., about 5 to about 35 nucleotides, about 5 to about 30 nucleotides, about 5 to about 25 nucleotides, about 5 to about 20 nucleotides, about 5 to about 15 nucleotides, about 5 to about 10 nucleotides, about 10 to about 40 nucleotides, about 10 to about 35 nucleotides, about 10 to about 30 nucleotides, about 10 to about 25 nucleotides, about 10 to about 20 nucleotides, about 10 to about 15 nucleotides, about 15 to about 40 nucleotides, about 15 to about 35 nucleotides, about 15 to about 30 nucleotides, about 15 to about 25 nucleotides, about 15 to about 20 nucleotides, about 20 to about 40 nucleotides, about 20 to about 35 nucleotides, about 20 to about 30 nucleotides, about 20 to about 25 nucleotides, about 25 to about 40 nucleotides, about 25 to about 35 nucleotides, about 25 to about 30 nucleotides, about 30 to about 40 nucleotides, about 30 to about 35 nucleotides, or about 35 to about 40 nucleotides) in length.

In some embodiments, the capture domain is positioned 3′ relative to the spatial barcode in the capture probe. In some embodiments of any of the methods provided herein, the capture probe further includes a unique molecular identifier, a cleavage domain (e.g., any of the exemplary cleavage domains described herein), one or more functional domains (e.g., a sequencing primer domain), or combinations thereof.

In some embodiments, the step of extending the 3′ end of the capture probe using the RNA bound by the capture domain as a template includes the use of a reverse transcriptase. In some embodiments, the step of generating the one or more nick(s) in the RNA hybridized to the extended capture probe is performed using RNAse H or a functional equivalent thereof. In some embodiments, the random-primed DNA synthesis is performed using DNA polymerase I, and optionally, a ligase (e.g., DNA ligase). In some embodiments, the end repair on the second strand DNA molecule hybridized to the extended capture probe to generate a blunt-ended double-stranded DNA product, is performed using a Klenow fragment and/or T4 DNA polymerase. In some embodiments, the step of adding a single adenosine (A) nucleotide to the 3′ end of the extended capture probe in the blunt-ended double-stranded DNA product is performed using Taq polymerase. In some embodiments, the step of ligating a double-stranded sequencing adaptor to the double-stranded DNA product is performed using a kinase (e.g., a polynucleotide kinase) and a DNA ligase.

In some embodiments, the capture probe further comprises a cleavage domain. Some embodiments of any of the methods described herein can further include, between the step of ligating the double-stranded sequencing adaptor to the double-stranded DNA product and the step of determining (i) all or a part of the sequence of the RNA or a complement thereof, or (ii) all or a part of the sequence of the spatial barcode or a complement thereof, and using the determined sequences of (i) and (ii) to identify the location of the RNA in the biological sample, a step of releasing the blunt-ended double-stranded DNA product from the array via cleavage of the cleavage domain.

In some embodiments of any of the methods described herein, the step of releasing the RNA from the biological sample comprises permeabilizing the biological sample (e.g., using any one or more of any of the permeabilization agents described herein and/or any of the physical conditions (e.g., heating) described herein.

In some embodiments of any of the methods described herein, the plurality of capture probes are affixed (i.e., attached) to an array. In some embodiments of any of the methods described herein, the array is a slide (e.g., a slide comprising beads or a slide comprising wells (e.g., microwells)). An array can also have one or more of any of the exemplary characteristics of arrays described herein.

In some embodiments, the step of determining (i) all or a part of the sequence of the RNA or a complement thereof, or (ii) all or a part of the sequence of the spatial barcode or a complement thereof, and using the determined sequences of (i) and (ii) to identify the location of the RNA in the biological sample comprises sequencing (i) all or a part of the sequence of the RNA or a complement thereof, or (ii) all or a part of the sequence of the spatial barcode or a complement thereof. In some embodiments, the sequencing can be performed using any of the exemplary sequencing methods described herein (e.g., high throughput sequencing). In some embodiments of any of the methods described herein, the RNA is mRNA. In some embodiments, the RNA is any of the other exemplary types of RNA described herein or known in the art.

In some embodiments of any of the methods described herein, the biological sample can be any of the exemplary biological samples described herein. For example, a biological sample can be a tissue sample or a tissue section. For example, the biological sample can be a fixed tissue section (e.g., a formalin-fixed paraffin-embedded (FFPE) tissue section, acetone fixed tissues section, methanol fixed tissue section, or a paraformaldehyde fixed tissue section) or a fresh, frozen tissue section.

Some embodiments of any of the methods described herein can further include one or both of staining and imaging the biological sample (e.g., using any of the exemplary stains and staining methods described herein and using any of the exemplary imaging methods described herein). In some embodiments of any of the methods described herein, the biological sample was previously stained and/or imaged (e.g., before the releasing in step (b)). In some embodiments, the staining can include the use of an optical label as described herein, including, but not limited to, fluorescent, radioactive, chemiluminescent, calorimetric, or colorimetric detectable labels. In some embodiments, staining can include the use of a fluorescent antibody directed to a target analyte (e.g., cell surface or intracellular proteins) in the biological sample. In some embodiments, staining can include the use of a chemical stain, such as hematoxylin and eosin (H&E) or periodic acid-schiff (PAS), or immunofluorescence (IF) techniques as known in the art. In some embodiments, significant time (e.g., days, months, or years) can elapse between staining and/or imaging the biological sample and performing any of the methods described herein.

### Reaction Mixtures and Kits

Also provided herein are reaction mixtures that include: an array comprising a plurality of capture probes affixed thereon, wherein a capture probe of the plurality comprises (i) a spatial barcode and (ii) a capture domain that binds specifically to an RNA; RNAse H; DNA polymerase I; DNA ligase; and a double-stranded sequencing adaptor.

Also provided herein are kits that include: an array comprising a plurality of capture probes, where a capture probe of the plurality comprises (i) a spatial barcode and (ii) a capture domain that binds specifically to an RNA; RNAse H; DNA polymerase I; DNA ligase; and a double-stranded sequencing adaptor.

In some embodiments of any of the reaction mixtures or kits described herein, the capture domain can be any of the capture domains described herein. In some embodiments, the capture domain can comprise a poly(T) sequence. In some embodiments, the capture domain does not comprise a poly(T) sequence. In some embodiments, the capture domain comprises a sequence that is substantially complementary to a contiguous sequence present in the RNA. The capture domain can be about 5 to about 40 nucleotides (e.g., or any of the subranges of this range described herein) in length.

In some embodiments, the capture domain is positioned 3′ relative to the spatial barcode in the capture probe. In some embodiments of any of the reaction mixtures or kits provided herein, the capture probe further includes a unique molecular identifier, a cleavage domain (e.g., any of the exemplary cleavage domains described herein), a sequencing primer domain, a universal sequence, or combinations thereof. In some embodiments, the sequencing primer domain is positioned at the 5′ end relative to the capture domain, the spatial barcode, the unique molecular identifier, the cleavage domain, the universal sequence, or any combination thereof. In some embodiments, the sequencing primer is affixed to the substrate.

In some embodiments, the sequencing primer domain is positioned 5′ relative to the capture domain and the spatial barcode. In some embodiments, a functional domain (e.g., a sequencing primer) domain is positioned 5′ relative to the capture domain, the spatial barcode, and the unique molecular identifier. In some embodiments, the sequencing primer domain is positioned 3′ relative to the cleavage domain. In some embodiments, the sequencing primer domain is on the 5′ end that is affixed to the substrate.

In some embodiments of any of the reaction mixtures or kits described herein, the plurality of capture probes are affixed (i.e., attached) to an array. In some embodiments of any of the reaction mixtures or kits described herein, the array is a slide (e.g., a slide comprising beads or a slide comprising wells (e.g., microwells)). An array can also have one or more of any of the exemplary characteristics of arrays described herein.

A double-stranded adaptor sequencing adaptor can be any suitable double-stranded sequencing adaptor that can be used to perform any of the exemplary sequencing methods described herein. In some embodiments, the double-stranded sequencing adaptor can include an Illumina sequencing adaptor sequence. In some embodiments, the double-stranded sequencing adaptor can include a first and a second strand that each can be about 15 to 45 nucleotides (e.g., any of the subranges of this range described herein) long. In some embodiments, the double-stranded sequencing adaptor can include a universal sequence.

In some embodiments of any of the reaction mixtures described herein, the reaction mixture can include an RNA from a biological sample (e.g., an mRNA or any of the other types of RNA described herein or known in the art).

Some embodiments of any of the reaction mixtures or kits described herein further include one or both of Taq polymerase and a polynucleotide kinase (PNK). Some embodiments of any of the reaction mixtures or kits described herein further include a reverse transcriptase. Some embodiments of any of the reaction mixtures or kits described herein further include one or both of Klenow fragment and T4 DNA polymerase.

Some embodiments of any of the kits or reaction mixtures described herein can further include one or more permeabilization reagents (e.g., one or more of any of the permeabilization reagents described herein), such as for example, a protease.

Some embodiments of any of the kits described herein can further include a staining agent. In some embodiments, a staining agent can include an optical label, e.g., a fluorescent, a radioactive, a chemiluminescent, a calorimetric, or a colorimetric detectable label. In some embodiments, a staining agent can be a fluorescent antibody directed to a target analyte (e.g., cell surface or intracellular proteins). In some embodiments, a staining agent can be a chemical stain, such as hematoxylin and eosin (H&E) or periodic acid-schiff (PAS).

Some embodiments of any of the kits described herein can further include instructions for performing any of the methods described herein.

### Embodiments

Embodiment 1 is a method of identifying a location of an RNA in a biological sample, the method comprising: (a) contacting the biological sample with an array comprising a plurality of capture probes, wherein a capture probe of the plurality of capture probes comprises a capture domain and a spatial barcode; (b) releasing the RNA from the biological sample, wherein the RNA released from the biological sample is specifically bound by the capture domain of the capture probe; (c) extending a 3′ end of the capture probe using the RNA specifically bound by the capture domain as a template, thereby generating an extended capture probe hybridized to the RNA; (d) generating one or more nick(s) in the RNA hybridized to the extended capture probe and performing random-primed DNA synthesis, thereby replacing the nicked RNA with a second strand DNA molecule hybridized to the extended capture probe; (e) performing end repair on the second strand DNA molecule hybridized to the extended capture probe to generate a blunt-ended double-stranded DNA product; (f) adding a single adenosine (A) nucleotide to the 3′ end of the extended capture probe in the blunt-ended double-stranded DNA product; (g) ligating a double-stranded sequencing adaptor to the double-stranded DNA product; and (h) determining (i) all or a part of the sequence of the RNA or a complement thereof, or (ii) all or a part of the sequence of the spatial barcode or a complement thereof, and using the determined sequences of (i) and (ii) to identify the location of the RNA in the biological sample.

Embodiment 2 is the method of embodiment 1, wherein the generating of the one or more nick(s) in step (d) is performed using RNAse H.

Embodiment 3 is the method of embodiment 1, wherein the random-primed DNA synthesis is performed using DNA polymerase I.

Embodiment 4 is the method of any one of embodiments 1-3, wherein the end repair in step (e) is performed using a Klenow fragment and/or T4 DNA polymerase.

Embodiment 5 is the method of any one of embodiments 1-4, wherein step (f) is performed using Taq polymerase.

Embodiment 6 is the method of any one of embodiments 1-5, wherein step (g) is performed using a kinase and a DNA ligase.

Embodiment 7 is the method of embodiment 6, wherein the kinase is a polynucleotide kinase (PNK).

Embodiment 8 is the method of any one of embodiments 1-7, wherein the RNA is a mRNA.

Embodiment 9 is the method of embodiment 8, wherein the capture domain comprises a poly(T) sequence.

Embodiment 10 is the method of any one of embodiments 1-9, wherein the capture domain is positioned 3′ relative to the spatial barcode in the capture probe.

Embodiment 11 is the method of any one of embodiments 1-10, wherein the capture probe further comprises a unique molecular identifier.

Embodiment 12 is the method of any one of embodiments 1-11, wherein the capture probe further comprises a cleavage domain.

Embodiment 13 is the method of embodiment 12, wherein the method further comprises, between steps (g) and (h), a step of releasing the blunt-ended double-stranded DNA product from the array via cleavage of the cleavage domain.

Embodiment 14 is the method of any one of embodiments 1-13, wherein the determining in step (h) comprises sequencing (i) all or a part of the sequence of the RNA or a complement thereof, or (ii) all or a part of the sequence of the spatial barcode or a complement thereof.

Embodiment 15 is the method of embodiment 14, wherein the sequencing is high throughput sequencing.

Embodiment 16 is the method of embodiment 15, wherein the high throughput sequencing comprises sequencing by ligation.

Embodiment 17 is the method of embodiment 15, wherein the high throughput sequencing comprises hybridization of labelled probes.

Embodiment 18 is the method of any one of embodiments 1-14, wherein the method further comprises creating a library for sequencing from the adaptor-ligated double-stranded DNA product.

Embodiment 19 is the method of embodiment 18, wherein the step of creating the library comprises amplification.

Embodiment 20 is the method of embodiment 19, wherein the amplification comprises polymerase chain reaction.

Embodiment 21 is the method of embodiment 20, wherein the method further comprises sequencing the library.

Embodiment 22 is the method of any one of embodiments 1-21, wherein the releasing in step (b), comprises permeabilizing the biological sample.

Embodiment 23 is the method of any one of embodiments 1-22, wherein the array is a slide.

Embodiment 24 is the method of embodiment 23, wherein the slide comprises beads.

Embodiment 25 is the method of embodiment 23, wherein the slide comprises wells.

Embodiment 26 is the method of any one of embodiments 1-25, wherein the biological sample is a tissue sample.

Embodiment 27 is the method of embodiment 26, wherein the tissue sample is a tissue section.

Embodiment 28 is the method of embodiment 27, wherein the tissue section is a fixed tissue section.

Embodiment 29 is the method of embodiment 28, wherein the fixed tissue section is a formalin-fixed paraffin-embedded tissue section.

Embodiment 30 is the method of embodiment 27, wherein the tissue section is a fresh, frozen tissue section.

Embodiment 31 is the method of any one of embodiments 1-30, wherein the method further comprising one or both of staining and imaging the biological sample.

Embodiment 32 is a reaction mixture comprising: an array comprising a plurality of capture probes, wherein a capture probe of the plurality comprises (i) a spatial barcode and (ii) a capture domain that binds specifically to an RNA; RNAse H; DNA polymerase I; DNA ligase; and a double-stranded sequencing adaptor.

Embodiment 33 is the reaction mixture of embodiment 32, wherein the capture domain is positioned 3′ of the spatial barcode in the capture domain.

Embodiment 34 is the reaction mixture of embodiment 32 or 33, wherein the capture probe further comprises a unique molecular identifier.

Embodiment 35 is the reaction mixture of any one of embodiments 32-34, wherein the capture probe further comprises a cleavage domain.

Embodiment 36 is the reaction mixture of any one of embodiments 32-35, wherein the reaction mixture further comprises an RNA from a biological sample.

Embodiment 37 is the reaction mixture of any one of embodiments 32-36, wherein the array is a slide.

Embodiment 38 is the reaction mixture of embodiment 37, wherein the slide comprises beads.

Embodiment 39 is the reaction mixture of embodiment 37, wherein the array comprises wells.

Embodiment 40 is the reaction mixture of any one of embodiments 32-39, wherein the reaction mixture further comprises one or both of Taq polymerase and a polynucleotide kinase (PNK).

Embodiment 41 is the reaction mixture of any one of embodiments 32-40, wherein the reaction mixture further comprises reverse transcriptase.

Embodiment 42 is the reaction mixture of any one of embodiments 32-41, wherein the reaction mixture further comprises one or both of Klenow fragment and T4 DNA polymerase.

Embodiment 43 is a kit comprising: an array comprising a plurality of capture probes, wherein a capture probe of the plurality comprises (i) a spatial barcode and (ii) a capture domain that binds specifically to an RNA; RNAse H; DNA polymerase I; DNA ligase; and a double-stranded sequencing adaptor.

Embodiment 44 is the kit of embodiment 43, wherein the capture domain is positioned 3′ of the spatial barcode in the capture domain.

Embodiment 45 is the kit of embodiment 43 or 44, wherein the capture probe further comprises a unique molecular identifier.

Embodiment 46 is the kit of any one of embodiments 43-45, wherein the capture probe further comprises a cleavage domain.

Embodiment 47 is the kit of any one of embodiments 43-46, wherein the array is a slide.

Embodiment 48 is the kit of embodiment 47, wherein the slide comprises beads.

Embodiment 49 is the kit of embodiment 47, wherein the array comprises wells.

Embodiment 50 is the kit of any one of embodiments 43-49, wherein the kit further comprises one or both of Taq polymerase and a polynucleotide kinase (PNK).

Embodiment 51 is the kit of any one of embodiments 43-50, wherein the kit further comprises reverse transcriptase.

Embodiment 52 is the kit of any one of embodiments 43-51, wherein the kit further comprises one or both of Klenow fragment and T4 DNA polymerase.

What is claimed is:

1. A method of identifying a location of an RNA in a biological sample, the method comprising:
(a) contacting the biological sample with an array comprising a plurality of capture probes, wherein a capture probe of the plurality of capture probes comprises a capture domain and a spatial barcode;
(b) releasing the RNA from the biological sample, wherein the RNA released from the biological sample is specifically bound by the capture domain of the capture probe;
(c) extending a 3′ end of the capture probe using the RNA bound by the capture domain as a template, thereby generating an extended capture probe hybridized to the RNA;
(d) generating one or more nick(s) in the RNA hybridized to the extended capture probe and performing random-primed DNA synthesis, thereby replacing the nicked RNA with a second strand DNA molecule hybridized to the extended capture probe;
(e) performing end repair on the second strand DNA molecule hybridized to the extended capture probe to generate a blunt-ended double-stranded DNA product;
(f) adding a single adenosine (A) nucleotide to the 3′ end of the extended capture probe in the blunt-ended double-stranded DNA product;
(g) ligating a double-stranded sequencing adaptor to the double-stranded DNA product; and
(h) determining (i) all or a part of the sequence of the RNA or a complement thereof, or (ii) all or a part of the sequence of the spatial barcode or a complement thereof, and using the determined sequences of (i) and (ii) to identify the location of the RNA in the biological sample.
